(19) United States
(12) Patent Application Publication
(10) Pub. No.: US 2019/0093214 A1
(43) Pub. Date: Mar. 28, 2019

(54) NATIVE OR UNCONTROLLED OXIDE REDUCTION BY A CYCLIC PROCESS OF PLASMA TREATMENT AND H* RADICALS

(71) Applicant: Applied Materials, Inc., Santa Clara, CA (US)

(72) Inventor: Sukti CHATTERJEE, San Jose, CA (US)

(21) Appl. No.: 16/053,083

(22) Filed: Aug. 2, 2018

Related U.S. Application Data
(60) Provisional application No. 62/561,993, filed on Sep. 22, 2017.

(51) Int. Cl. C23C 16/02; C12Q 1/6869; C23C 16/30

(52) U.S. Cl. CPC C23C 16/02; C23C 16/30; C12Q 1/6869

(57) ABSTRACT

Methods are disclosed to provide arrays of substantially oxide-free or uncontrolled oxide-free structures, such as titanium nanotubes or microwells. In one aspect, the method includes plasma treating the structure having an oxide layer thereon to weaken the bonds in the oxide layer and then bombarding the oxide layer having weakened bonds with hydrogen radicals to remove the oxide layer to form a titanium layer. The cyclic plasma treatment and hydrogen radical exposure processes are generally repeated until the oxide layer is removed from the structure. Arrays of titanium structures manufactured according to the described methods are well controlled and have improved device performance since the oxide layer has been removed and the signal-to-noise ratio of the device has been optimized for improved sensing.

### CROSS-REFERENCE TO RELATED APPLICATIONS

This application claims benefit of U.S. Provisional Patent Application Ser. No. 62/561,993, filed on Sep. 22, 2017, which is herein incorporated by reference in its entirety.

### BACKGROUND

### Field

Aspects disclosed herein relate to methods of manufacturing arrays of substantially oxide-free structures, such as titanium nanotubes or microwells.

### Description of the Related Art

Titanium oxide (TiOₓ) is useful for various physical and chemical functions, including for use as a gas, ion, or biological species-sensing material. Accordingly, TiOₓ is being used for various biometric sensing applications, such as in a phosphate sensor for DNA sequencing. Conventionally, porous TiOₓ films are formed on a substrate by anodization methods, such as anodic oxidation of a titanium sheet in an aqueous solution containing hydrofluoric (HF) acid.

One problem with conventionally manufactured TiOₓ structure arrays, however, is that the formed structures have unwanted oxides, such as native oxides or otherwise uncontrolled oxides, on the surfaces thereof. The oxides are generally the result of anodization or other process steps. The native oxides negatively affect device uniformity and performance, for example, by increasing a signal-to-noise ratio in a biological species-sensing device. Additionally, conventionally manufactured arrays are not highly ordered.

Therefore, there is a need in the art for methods of reducing or eliminating oxides on the arrays of TiOₓ structures, such as nanotubes or microwells, or other TiOₓ films.

### SUMMARY

Methods are disclosed to provide arrays of substantially oxide-free structures, such as titanium nanotubes or microwells. In one aspect, the method includes plasma treating the structure having an oxide layer thereon to weaken the bonds in the oxide layer and then bombarding the oxide layer having weakened bonds with hydrogen radicals to reduce the oxide layer to a titanium layer. The cyclic plasma treatment and hydrogen radical exposure processes are generally repeated until the oxide layer is removed from the structure. Arrays of titanium structures manufactured according to the described methods are well controlled and have improved device performance since the oxide layer has been removed and the signal-to-noise ratio of the device has been optimized for improved sensing.

In one aspect, a method for reducing oxides is disclosed. The method includes positioning a substrate having an array of metal structures formed thereon in a process chamber, the array of metal structures having an oxide layer formed thereon, plasma treating the oxide layer to form a plasma-treated oxide layer, and exposing the plasma-treated oxide layer to hydrogen or argon/hydrogen radicals to remove the plasma-treated oxide layer.

In another aspect, a method for reducing oxides is disclosed. The method includes positioning a substrate having an array of titanium oxide structures formed thereon in a first process chamber, the array of titanium oxide structures having an oxide layer formed thereon, plasma treating the oxide layer to form a plasma-treated oxide layer having weakened titanium-oxygen bonds in the first process chamber, transferring the substrate to a second process chamber, and exposing the plasma-treated oxide layer to hydrogen or argon/hydrogen radicals to remove the plasma-treated oxide layer in the second process chamber.

In yet another aspect, a titanium oxide structure is disclosed. The titanium oxide structure includes a complementary metal oxide stack layer having a sensor therein, a titanium nitride layer disposed over the complementary metal oxide stack layer, a titanium layer disposed over the titanium nitride layer, and a plasma-treated oxide layer disposed over the titanium layer, the plasma-treated oxide layer having weakened titanium-oxygen bonds therein.

### BRIEF DESCRIPTION OF THE DRAWINGS

So that the manner in which the above recited features of the present disclosure can be understood in detail, a more particular description of the disclosure, briefly summarized above, may be had by reference to aspects, some of which are illustrated in the appended drawings. It is to be noted, however, that the appended drawings illustrate only exemplary aspects and are therefore not to be considered limiting of its scope. The present disclosure may admit to other equally effective aspects.

FIG. 1 is a perspective side view of a portion of a substrate having an array of titanium structures formed thereon.

FIG. 2 is a top view of the array of titanium structures of FIG. 1.

FIG. 3 is a process flow for reducing oxides from a TiOₓ structure.

FIGS. 4A-4C depict cross-sectional views of a titanium structure formed according to a process flow disclosed herein.

FIG. 5 is a substrate processing system that may be used to perform a process flow disclosed herein.

To facilitate understanding, identical reference numerals have been used, where possible, to designate identical elements that are common to the figures. It is contemplated that elements and features of one aspect may be beneficially incorporated in other aspects without further recitation.

### DETAILED DESCRIPTION

Methods are disclosed to provide arrays of substantially oxide-free structures, such as titanium nanotubes or microwells. In one aspect, the method includes plasma treating the structure having an oxide layer thereon to weaken the bonds in the oxide layer and then bombarding the oxide layer having weakened bonds with hydrogen radicals to remove the oxide layer. The cyclic plasma treatment and hydrogen radical exposure processes are generally repeated until the oxide layer is removed from the structure. Arrays of titanium structures manufactured according to the described methods are well controlled and have improved device performance since the oxide layer has been removed and the signal-to-noise ratio of the device has been optimized for improved sensing.

Methods described herein will refer to reduction of a TiOₓ native oxide layer of titanium nanotubes or microwells as an example. However, it is also contemplated that the described methods are useful to reduce native oxides on any structures, such as other metal structures or carbon structures. The described methods are also useful for reducing native oxides on films, such as high quality titanium dioxide (TiO₂) films deposited by atomic layer deposition (ALD). Additionally, the described methods are useful to remove any uncontrolled or otherwise unwanted oxides.

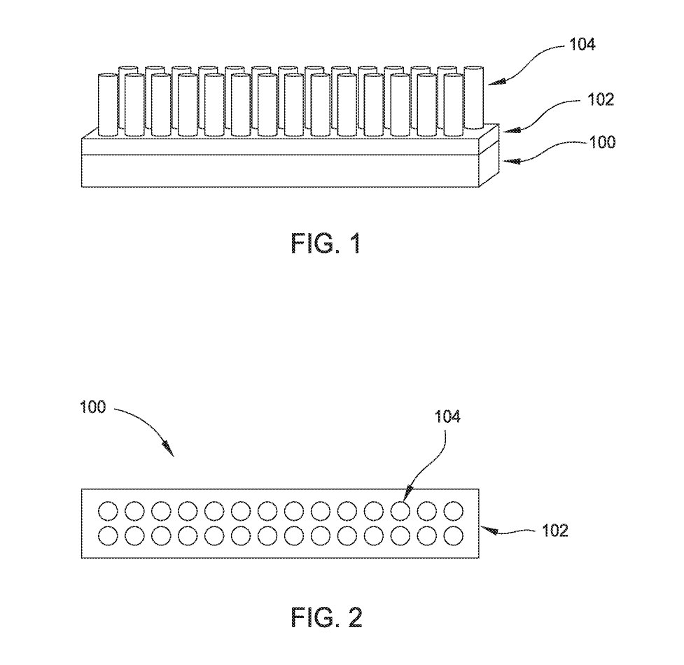

FIG. 1 is a perspective side view of a portion of a substrate 100 having an array of titanium structures 104 formed thereon. FIG. 2 is a top view of the array of titanium structures 104 of FIG. 1. As shown in FIG. 1, a TiOₓ barrier layer 102 is disposed over the substrate 100. An array of titanium structures 104 is formed on the TiOₓ barrier layer 102. The TiOₓ barrier layer 102 provides a bottom surface of the titanium structures 104.

The substrate 100 is generally any substrate having a porous layer thereon. A porous layer is generally any layer having natural pores thereon. In one aspect, the substrate 100 has a porous titanium layer thereon. Some examples of porous titanium, which is useful for gas and biological-species sensing, include uniform pore structures such as nanotubes and microwells, bimodal pore structures, gradient pore structures, honeycomb structures, and closed-pore structures. As shown in FIG. 1, the titanium structures 104 are nanotubes.

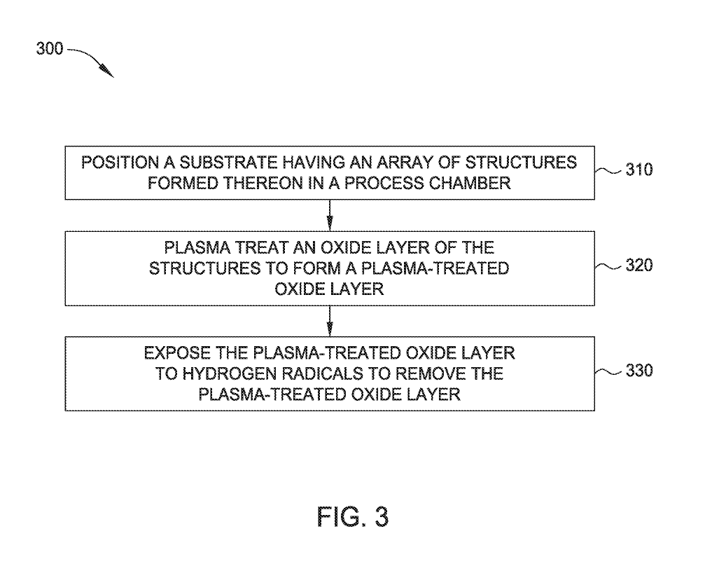

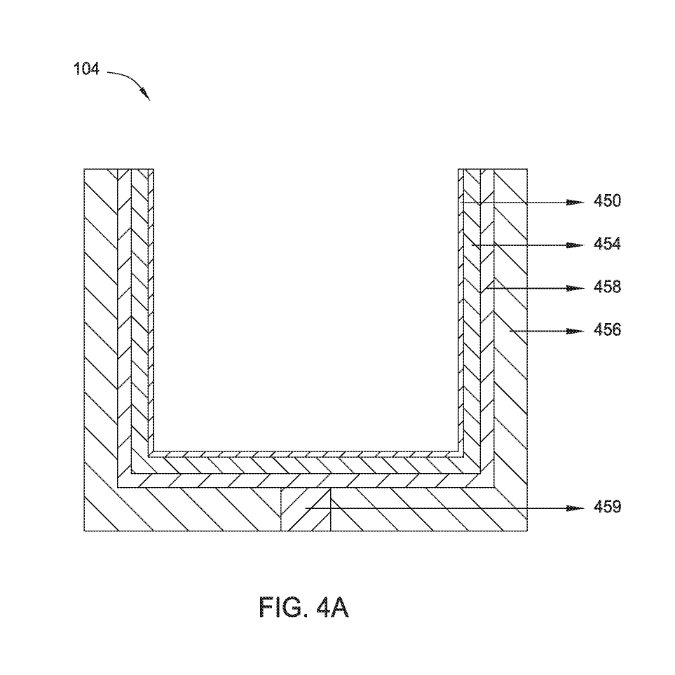

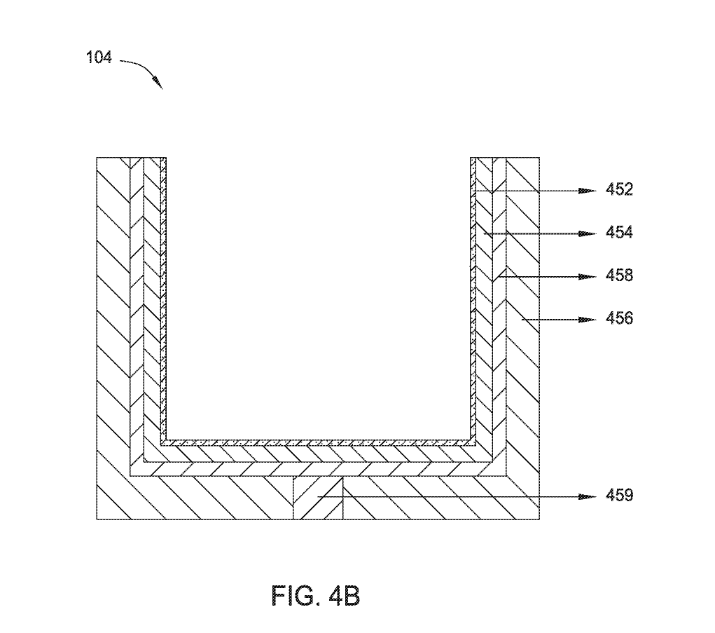

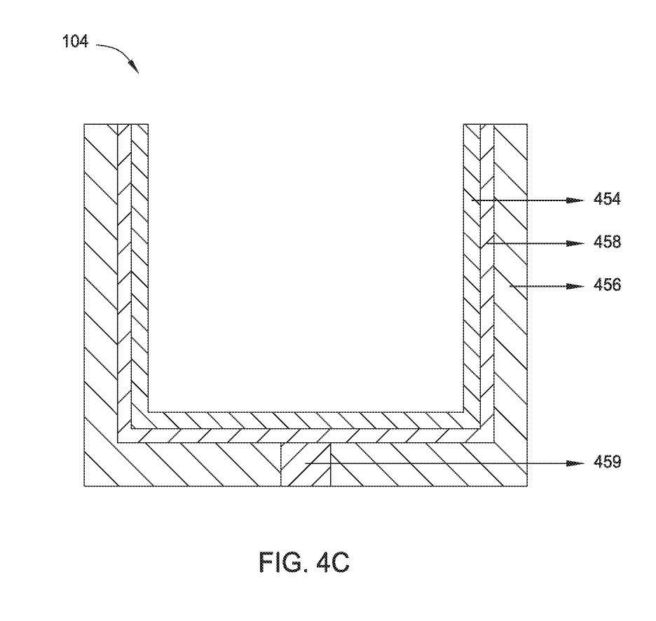

FIG. 3 is a process flow 300 for reducing oxides from a TiOₓ structure. FIGS. 4A-4C depict cross-sectional views of a titanium structure 104 formed according to process flows disclosed herein, such as at various operations of the process flow 300.

The process flow 300 begins at operation 310 by positioning a substrate 100 having an array of titanium structures 104, shown as TiOₓ structures as an example, formed thereon in a process chamber.

Prior to operation 310, the substrate 100 having the array of titanium structures 104 formed thereon is generally formed by any suitable method. In one aspect, an array of titanium structures 104 is formed by depositing a titanium layer, such as titanium thin film, over the substrate 100 and anodizing the titanium layer in an HF acid solution to form the titanium structures 104, such as TiOₓ structures. As shown in FIG. 4A, the formed titanium structure 104 is a microwell, which includes a native oxide layer 450 on the surface thereof, as an example. However, the process flow 300 is useful for removing generally any oxides from a substrate surface. In some aspects, the formed titanium structure 104 also includes various additional layers, including but not limited to, a complementary metal-oxide semiconductor (CMOS) stack 456 having a biometric sensor 459 therein, a titanium nitride (TiN) layer 458 and a titanium (Ti) layer 454. In one aspect, the TiN layer 458 is disposed over the CMOS stack 456 and the Ti layer 454 is disposed over the TiN layer 458. In another aspect, for example, the TiN layer 458 is disposed on an in contact with the CMOS stack 456, and the Ti layer 454 is disposed on and in contact with the TiN layer 458. As discussed above, the native oxide layer 450 affects device uniformity and performance.

At operation 320, the substrate 100 is exposed to a plasma treatment process to weaken the bonds in the native oxide layer 450, such as titanium-oxygen (Ti—O) bonds in a TiOₓ layer, to form plasma-treated oxide layer 452, as shown in FIG. 4B. In one aspect, weakening the bonds includes physically damaging the native oxide layer 450 such that a lower-energy, for example between about 1 and about 3 electron volts (eV), is required for subsequent breaking the bonds during subsequent exposure to hydrogen radicals. The plasma treatment physically damages or otherwise weakens the Ti—O bonds of the native oxide layer 450 so that the plasma-treated oxide layer 452 is prepared for subsequent reduction and removal.

At operation 330, the substrate is exposed to, or bombarded with, hydrogen radicals to remove the plasma-treated oxide layer 452. Since the plasma-treated oxide layer 452 has already been plasma-treated to weaken the Ti—O bonds of the layer, a low-energy hydrogen exposure can be used to remove the plasma-treated oxide layer 452 by reacting the hydrogen radicals with the weakly bonded Ti—O molecules. More specifically, the hydrogen radicals react with the oxide in the plasma-treated oxide layer 452 and cause an oxide reduction and formation of products, such as water (H₂O) and titanium hydrides. Using low-energy hydrogen selectively removes the plasma-treated oxide layer 452 and thus reduces the potential for damage to the other layers on the substrate.

In some aspects, only a portion of the plasma-treated oxide layer 452 will be removed during operation 330. Accordingly, operation 320 and operation 330 are generally repeated any number of times until the native oxide layer 450 is removed and titanium structures 104, which are substantially oxide free, are formed, as shown in FIG. 4C. Optionally, the substrate 100 is cooled down after operation 330 and prior to operation 320 being repeated.

In one aspect, plasma treating the substrate 100 at operation 320 includes a low energy plasma treatment at a plasma power of 13.56 Megahertz (MHz). The plasma precursor is generally an unreactive gas, including but not limited to an inert gas, such as argon (Ar) and/or helium (He). The flow rate of the plasma precursor is between about 10 standard cubic centimeters per minute (sccm) and about 50 sccm. The radio frequency (RF) power is between about 200 watts (W) and about 700 W. The process chamber pressure is between about 5 millitorr (mTorr) and about 60 mTorr. The substrate is generally at a low temperature, for example, about room temperature (e.g., between about 20 degrees Celsius (° C.) and about 25° C.).

In one aspect, exposing the substrate 100 to hydrogen radicals to remove the plasma-treated oxide layer 452 at operation 330 includes a hot wire chemical vapor deposition (HWCVD) process. The HWCVD process generally includes providing hydrogen (H₂) gas into a process chamber, such as an HWCVD chamber, at any suitable flow rate, heating one or more filaments disposed in the process chamber to a temperature sufficient to dissociate the H₂ gas and provide at least a portion of the energy for facilitating subsequent removal of at least a portion of the plasma-treated oxide layer 452, and exposing the substrate 100 to the dissociated H₂ gas to remove at least some of the plasma-treated oxide layer 452. The one or more filaments are generally heated to a temperature between about 1,200° C. and about 1,700° C. The temperature of a substrate heater is generally low, for example, between about 200° C. and about 400° C. The flow rate of the H₂ gas is generally between about 100 sccm and about 500 sccm, for example, about 400 sccm. The process chamber pressure is generally between about 0.1 torr (T) and about 1.0 T, for example, about 0.5 T. The duration of the HWCVD process is generally between about 50 seconds and about 4 hours, for example between about 100 seconds and about 200 seconds, such as about 120 seconds.

In another aspect, exposing the substrate 100 to hydrogen radicals to remove the plasma-treated oxide layer 452 at operation 330 includes introducing hydrogen radicals to a process chamber from a remote microwave or radiofrequency (RF) plasma source (RPS).

In one aspect, the substrate 100 is optionally cooled down for between about 50 seconds and about 200 seconds, such as 120 seconds, after operation 330 and prior to operation 320 being repeated.

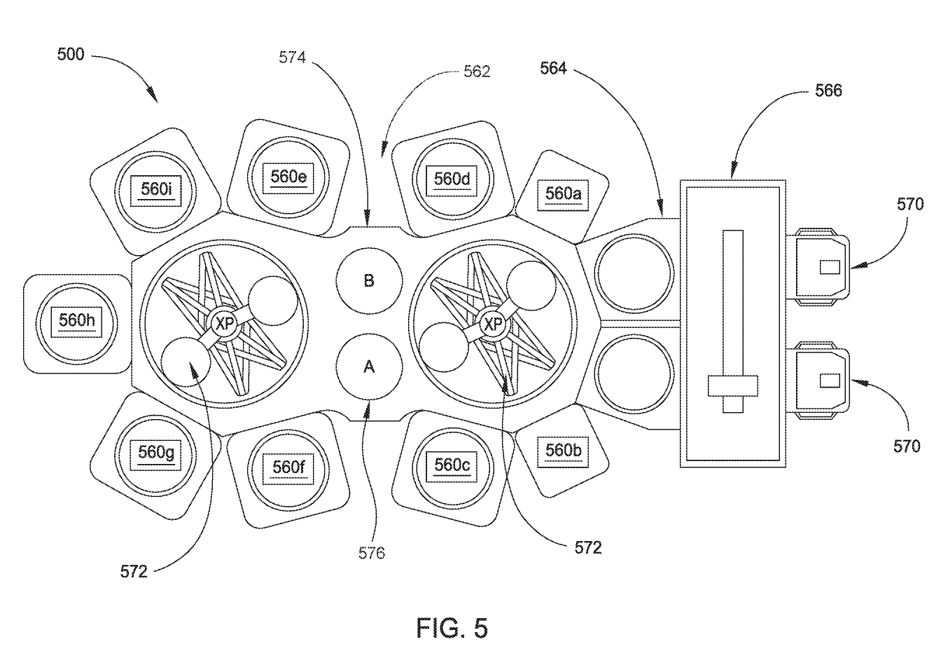

FIG. 5 is a schematic top view of a substrate processing system 500 that may be used to perform a process flow disclosed herein, such as a system available from Applied Materials, Inc., Santa Clara, Calif. The substrate processing system 500 generally includes a plurality of processing chambers 560a-560i, a transfer chamber 562, a load lock chamber 564, and a factory interface 566. The transfer chamber 562 is coupled to the load lock chamber 564 and the processing chambers 560a-i. The load lock chamber 564 is coupled between the factory interface 566 and the transfer chamber 562.

The factory interface 566 is maintained at a substantially atmospheric pressure and includes one or more robots 572 for transferring substrates between cassettes 570 coupled to the factory interface 566 and the load lock chamber 564. Robots 572 are configured to transfer substrates into the substrate processing chambers 560 for processing. A third chamber 574, such as a buffer chamber with substrate cooling stations 576, is positioned between the robots 572.

As illustrated in FIG. 5, the substrate processing system 500 includes nine processing chamber 560a-i. However, nine processing chambers 560a-i is for illustrative purposes. The substrate processing system 500 generally includes any suitable number of processing chambers. Each substrate processing chamber 560a-i can be outfitted to perform a substrate processing operation such as dry etch processes, cyclical layer deposition (CLD), ALD, chemical vapor deposition (CVD), HWCVD, physical vapor deposition (PVD), pre-clean, substrate degas, substrate orientation, and other substrate processes. Examples of such process chambers are available from Applied Materials, Inc., Santa Clara, Calif.

According to aspects described herein, the system 500 generally includes at least three processing chambers. For example, a first substrate processing chamber 560a is a pre-clean chamber configured to plasma treat the substrate 100 as described above at operation 320, and a second process chamber 560f is an HWCVD chamber configured to expose the substrate 100 to hydrogen radicals as described above at operation 330. Additionally, the third chamber 574 is a buffer chamber having substrate cooling stations therein for cooling the substrate 100 between repetitions of operations 320 and 330.

As shown in FIGS. 1 and 2, the array of titanium structures 104 formed by process flows described herein is well ordered. Well ordered generally means that the titanium structures 104 of the array are arranged in an orderly and substantially uniform way. In the example shown in FIG. 4C, the formed titanium structure 104 is a microwell having a sensor 459, such as a phosphate sensor for DNA sequencing. The formed titanium structure 104 is useful for applications, such as photocatalysis, solar cells, electrochromic devices, biomedical coatings, drug delivery, gas sensing, biological species sensing, and other biomedical applications. Since the native oxide layer 450 has been removed from the microwell, the signal-to-noise ratio is optimized, for example improved by 30%, thus increasing device sensitivity, and the ability of the sensor 459 to be used for the various applications.

Benefits of the present disclosure include formation of well-ordered arrays of titanium structures, such as nanotubes or microwells, which provide improved performance because the oxides on surfaces thereof have been removed to form a titanium layer. Additionally, the cyclic process of plasma treatment and exposure to hydrogen radicals provides a method of removing oxides at lower temperatures, which can be achieved in existing process chambers, such as pre-clean chambers and HWCVD chambers, without having to retrofit the chambers for higher temperature components.

While the foregoing is directed to aspects of the present disclosure, other and further aspects of the disclosure may be devised without departing from the basic scope thereof, and the scope thereof is determined by the claims that follow.

What is claimed is:

1. A method for reducing oxides, comprising:
positioning a substrate having an array of metal structures formed thereon in a process chamber, the array of metal structures having an oxide layer formed thereon;
plasma treating the oxide layer to form a plasma-treated oxide layer; and
exposing the plasma-treated oxide layer to hydrogen radicals to remove the plasma-treated oxide layer.

2. The method of claim 1, wherein the array of metal structures comprise titanium structures.

3. The method of claim 1, further comprising:
repeating the plasma treating the oxide layer to form the plasma-treated oxide layer and the exposing the plasma-treated oxide layer to hydrogen radicals to remove the plasma-treated oxide layer.

4. The method of claim 1, wherein plasma treating the oxide layer comprises:
introducing a plasma precursor into the process chamber, wherein the plasma precursor comprises at least one of argon and helium.

5. The method of claim 4, wherein a flow rate of the plasma precursor is between about 10 sccm and about 50 sccm an RF power is between about 200 W and about 700 W, and a process chamber pressure is between about 5 mTorr and about 60 mTorr.

6. The method of claim 1, wherein exposing the plasma-treated oxide layer to hydrogen radicals comprises:
performing a hot wire chemical vapor deposition process.

7. The method of claim 6, wherein the hot wire chemical vapor deposition process comprises:
providing hydrogen gas into the process chamber;
heating one or more filaments disposed in the process chamber to a temperature sufficient to dissociate the hydrogen gas; and
exposing the plasma-treated oxide layer to the dissociated hydrogen gas to remove at least a portion of the plasma-treated oxide layer.

8. The method of claim 7, wherein the one or more filaments are heated to a temperature between about 1,200° and about 1,700° C., a flow rate of the hydrogen gas is between about 100 sccm and about 500 sccm, and a process chamber pressure is between about 0.1 T and about 1.0 T.

9. The method of claim 8, wherein the flow rate of the hydrogen gas is about 400 sccm and the process chamber pressure is about 0.5 T.

10. The method of claim 1, wherein exposing the plasma-treated oxide layer to hydrogen radicals comprises introducing hydrogen gas into the process chamber from a remote plasma source.

11. A method for reducing oxides, comprising:
positioning a substrate having an array of titanium oxide structures formed thereon in a first process chamber; the array of titanium oxide structures having an oxide layer formed thereon;
plasma treating the oxide layer to form a plasma-treated oxide layer having weakened titanium-oxygen bonds in the first process chamber;
transferring the substrate to a second process chamber; and
exposing the plasma-treated oxide layer to hydrogen radicals to remove the plasma-treated oxide layer in the second process chamber.

12. The method of claim 11, wherein the first process chamber is a pre-clean chamber and the second process chamber is an HWCVD chamber.

13. The method of claim 11, further comprising:
repeating the plasma treating the oxide layer to form the plasma-treated oxide layer having weakened titanium-oxygen bonds in the first process chamber and the exposing the plasma-treated oxide layer to hydrogen radicals to remove the plasma-treated oxide layer.

14. The method of claim 13, further comprising:
transferring the substrate to a third chamber; and
cooling down the substrate in the third chamber before repeating the plasma treating and the exposing the plasma-treated oxide layer to hydrogen radicals.

15. The method of claim 11, wherein plasma treating the oxide layer comprises:
introducing a plasma precursor into the first process chamber, wherein the plasma precursor comprises at least one of argon and helium.

16. The method of claim 11, wherein exposing the plasma-treated oxide layer to hydrogen radicals comprises:
performing a hot wire chemical vapor deposition process; comprising:
providing hydrogen gas into the second process chamber;
heating one or more filaments disposed in the second process chamber to a temperature sufficient to dissociate the hydrogen gas; and
exposing the plasma-treated oxide layer to the dissociated hydrogen gas to remove at least a portion of the plasma-treated oxide layer.

17. The method of claim 16, wherein the one or more filaments are heated to a temperature between about 1,200° C. and about 1,700° C., a flow rate of the hydrogen gas is between about 100 sccm and about 500 sccm, and a process chamber pressure is between about 0.1 T and about 1.0 T.

18. A titanium oxide structure, comprising:
a complementary metal oxide stack layer having a sensor therein;
a titanium nitride layer disposed over the complementary metal oxide stack layer;
a titanium layer disposed over the titanium nitride layer; and
a plasma-treated oxide layer disposed over the titanium layer, the plasma-treated oxide layer having weakened titanium-oxygen bonds therein.

19. The titanium oxide structure of claim 18, wherein the sensor is a biometric sensor.

20. The titanium oxide structure of claim 19, wherein the titanium nitride layer is disposed on an in contact with the complementary metal oxide stack layer, the titanium layer is disposed on and in contact with the titanium nitride layer, and the plasma-treated oxide layer is disposed on and in contact with the titanium layer.
